(19) United States
(12) Patent Application Publication
(10) Pub. No.: US 2007/0260266 A1
(43) Pub. Date: Nov. 8, 2007

(54) LITHOTRIPSY COMPATIBLE WIRE BASKET

(75) Inventor: John A. Karpiel, Winston-Salem, NC (US)

Correspondence Address: BRINKS HOFER GILSON & LIONE/CHICAGO/COOK, PO BOX 10395, CHICAGO, IL, 60610, US

(73) Assignee: COOK INCORPORATED, Bloomington, IN (US)

(21) Appl. No.: 11/743,732

(22) Filed: May 3, 2007

Related U.S. Application Data
(60) Provisional application No. 60/797,242, filed on May 3, 2006.

(51) Int. Cl. A61B 17/22

(52) U.S. Cl. 606/127

(57) ABSTRACT

A medical device for performing lithotripsy on a foreign body within a patient. The medical device generally includes a wire basket constructed from distal portions of a plurality of multifilament wires. The distal portions of the plurality of multifilament wires each follow a generally helical path to give the wire basket a helical configuration. A method of performing lithotripsy is also provided using the medical device.

### CROSS-REFERENCE TO RELATED APPLICATIONS

This application claims the benefit of U.S. Provisional Application Ser. No. 60/797,242, filed on May 3, 2006, entitled “LITHOTRIPSY COMPATIBLE WIRE BASKET,” the entire contents of which are incorporated herein by reference.

### FIELD OF THE INVENTION

The present invention relates generally to medical devices for retrieval of foreign bodies, and more particularly relates to wire baskets suitable for performing lithotripsy.

### BACKGROUND OF THE INVENTION

Lithotripsy is a technique used to break up stones that form in the kidney, bladder, ureters, or gall bladder. There are several ways of performing lithotripsy, although the most common is shock wave lithotripsy which is performed outside the body (extracorporeal). The shock waves are focused on the stone and break the stone into tiny pieces, which may be passed out of the body naturally or through the use of retrieval baskets. Shock wave lithotripsy may also be performed within the body, as can other forms of endoscopic lithotripsy such as laser lithotripsy.

One other form of lithotripsy includes the use of wire baskets which are deployed to capture and engage the stone, whereupon the basket is collapsed or otherwise manipulated to crush the stone. These lithotripsy-compatible wire baskets must meet certain requirements which often conflict. In particular, the wire baskets must be flexible enough to traverse the anatomy, strong enough to open within a duct, and strong enough to crush stones.

One exemplary wire basket is disclosed in U.S. Pat. No. 5,330,482, the disclosure of which is hereby incorporated by reference in its entirety. Generally, the wire basket utilizes multifilament wire having an inner wire surrounded by a number of outer wires, thereby achieving the strength necessary for crushing stones. Despite these and other improvements, there remains a need for improvement of wire baskets capable of performing lithotripsy.

### BRIEF SUMMARY OF THE INVENTION

The present invention provides an improved medical device for performing lithotripsy on a foreign body within a patient. One embodiment of the medical device generally includes a wire basket constructed from a plurality of multifilament wires. The plurality of multifilament wires each follow a generally helical path to give the wire basket a helical configuration. The wire basket is configured to engage and crush the foreign body. This construction improves the capture and crushing of foreign bodies such as stones.

According to more detailed aspects of the present invention, the distal portions of the plurality of multifilament wire are joined at a first location, and are also joined at a second location axially spaced from the first location, the wire basket being defined between the first and second locations. The distal tip of the wire basket is formed at the second location, and preferably the wires are joined at the second location by solder forming an atraumatic distal tip. The wire basket is operable between a collapsed configuration for introduction into the patient and an expanded configuration for capturing the foreign body.

According to even more detailed aspects, each multifilament wire preferably includes at least three wires stranded together, and most preferably includes an inner wire surrounded by a plurality of outer wires which are stranded together. The inner wire is preferably constructed of a shaped memory material such as nitinol, and is designed to form the expanded configuration of the wire basket. The outer wires are preferably constructed of stainless steel, although they may also be constructed of a shaped memory material.

Another embodiment of a medical device constructed in accordance with the teachings of the present invention generally includes a wire assembly configured to be inserted into the patient in a collapsed configuration and then expand to an expanded configuration within the patient. The wire assembly includes a plurality of multifilament wires configured to engage and crush a foreign body. Each of the multifilament wires follows a spiral path in the expanded configuration. In this manner, the plurality of multifilament wires defines a helical wire basket that is configured to capture the foreign body in the expanded configuration.

Yet another embodiment of a medical device constructed in accordance with the teachings of the present invention generally provides a method of performing lithotripsy on a foreign body within a bodily lumen of a patient. A medical device is provided which generally includes a wire basket and a control member, the wire basket constructed from a plurality of multifilament wires which follow a generally helical path to give the wire basket a helical configuration. A delivery cannula is also provided which defines a delivery lumen, the control member being slidably disposed within the delivery lumen such that the wire basket is operable between a collapsed configuration within the delivery lumen and an expanded configuration outside of the delivery lumen. The wire basket and delivery cannula are positioned proximate the foreign body in the collapsed configuration. The control member is translated relative to the delivery cannula such that the wire basket assumes the expanded configuration. The control member is manipulated to capture the foreign body within the wire basket from a side of the wire basket. The wire basket is caused to assume its collapsed configuration to crush the foreign body.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a side view of a medical device constructed in accordance with the teaching of the present invention;

FIG. 2 is a side view depicting a multifilament wire forming part of the medical device of FIG. 1;

FIG. 3 is a cross-sectional view of the wire depicted in FIG. 2;

FIG. 4 is a perspective view of the medical device of FIG. 1;

FIG. 5 is a perspective view of another embodiment of a medical device constructed in accordance with the teachings of the present invention; and

FIG. 6 is a side view of a handle forming a portion of the medical device of FIG. 1.

### DETAILED DESCRIPTION OF THE INVENTION

Turning now to the figures, FIG. 1 depicts a medical device 20 constructed in accordance with the teachings of the present invention. The medical device 20 is constructed for performing lithotripsy on a foreign body within a patient, such as a stone located in the kidney, bladder, ureters, or gall bladder. The medical device 20 generally includes a wire basket 22 constructed from a plurality of multifilament wires 24. The multifilament wires 24 provide the flexibility to traverse the anatomy, as well as the stiffness to open within a duct and the strength to crush stones.

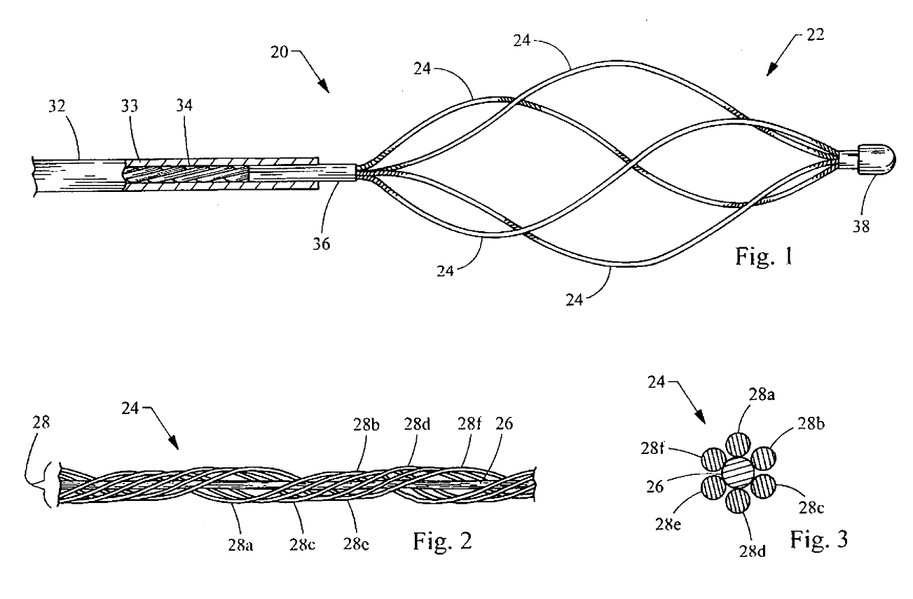

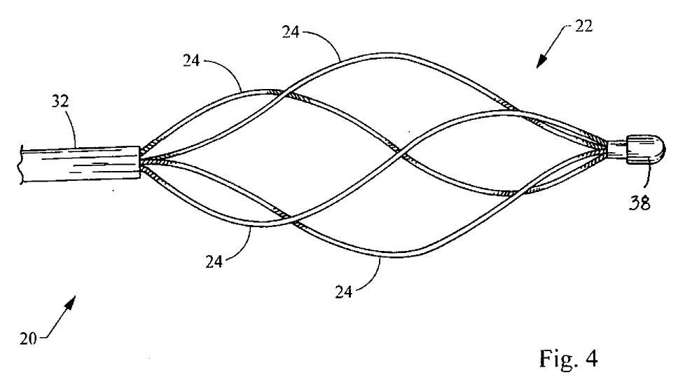

As best seen in FIGS. 2 and 3, each of the multifilament wires 24 is constructed of a plurality of solid wires, namely an inner wire 26 surrounded by a stranding 28 of six stranded wires 28a-f. The spacing between the stranding 28, as shown in FIG. 2, is for illustration purposes to show the relative orientation of the stranding, and does not occur in the wire 24 as actually constructed. FIG. 3 shows a cross-sectional view of the composite construction of multifilament wire 24. Generally, the inner wire 26 has a diameter of 0.005 inches and surrounding wires 28a-f each have a diameter of about 0.004 inches, giving an overall diameter to the wire 24 of about 0.013 inches. While this construction is preferred, it will be recognized by those skilled in the art that the multifilament wire 24 may vary in size, shape and construction. For example, the multifilament wire 24 may simply include a plurality of solid wires stranded together, braided, twisted, or joined by other known or future developed interconnection methods. Further details and variations of the multifilament wire 24 may be found in commonly owned U.S. Pat. No. 5,330,482.

Turning back to FIG. 1, the wire basket 22 is formed by distal portions of the plurality of multifilament wires 24. In particular, the wires are joined at a first location 36 (also referred to herein as first joint 36), and again joined at a second location 38 (“second joint 38”) which is axially spaced from the first location 36. Preferably, the plurality of multifilament wires 24 are joined by soldering or welding, which permits the second joint 38 to form the distal tip of the device 20. The distal tip formed by second joint 38 is atraumatically shaped to prevent damage to the patient's anatomy.

The wire basket 22 is preferably introduced into the body of the patient through a delivery cannula 32 which defines a delivery lumen 33. The delivery cannula 32 may be reinforced at its distal end, although a specially reinforced lithotripsy cable, such as the Wilson Cook® Conquest TTC™, is preferably introduced over the delivery cannula 32 for providing additional support during retraction of the wire basket 22 to collapse the basket 22 around the foreign body and crush the same.

The proximal portions of the plurality of multifilament wires 24 (i.e. proximal to first joint 36) are twisted together to form a control member 34 which extends proximally through the delivery lumen 33. It will be recognized that the control member 34 need not be constructed from the plurality of multifilament wires 24, but may rather comprise a separate member such as a solid rod which is connected to the wire basket 22 through the first joint 36. Similarly, other methods or structures may be used for forming the joints 36, 38, including the use of metal bands or clamps, whereby the control member 34 and wires 24 may be banded together at first joint 36. Accordingly, the control member 34 may be constructed out of a number of different materials including thermoplastics such as polyimide.

Manipulation of the control member 34 relative to the delivery cannula 32 provides control over the wire basket 22. Generally, the wire basket 22 may be withdrawn proximally within the delivery lumen 33 to define a collapsed configuration for introduction into the patient. The delivery cannula 32 may then be retracted (or the control member 34 extended) to expose the wire basket 22, whereby it transitions to an expanded configuration for capturing the foreign body, as shown in FIG. 1. FIG. 6 depicts a handle 40 for controlling the relative movement of the control member 34 relative to the delivery cannula 32. The handle 40 includes a first member 42 slidably connected to a second member 44. The first handle member 42 is connected to the control member 34, while the second handle member 44 is connected to the cannula 32 via a sleeve 46. Preferably, the first and second handle members 42, 44 define one or more rings for easy grasping and manipulation by the medical professional. A fitting 48 may optionally be provided to supply a fluid or other medium to the delivery cannula 32. In this manner, movement of the first handle member 42 relative to the second handle member 44 controls the relative positions of the control member 34 and delivery cannula 32, and hence operation of the wire basket 22.

To repeatedly achieve the expanded configuration of the wire basket 22, at least one of the individual wires 26, 28a-f is constructed of a shaped memory material, and preferably a superelastic alloy such as nitinol (a nickel titanium alloy). Most preferably, the inner wire 26 is formed of nitinol while the outer wires 28a-f are formed of stainless steel. In this manner, each multifilament wire 24 is provided with shape memory functionality, while also permitting soldering of the wires 24 due to the stainless steel stranding 28. However, it will be recognized that all or a portion of the outer wires 28a-f may also be constructed of a shape memory material (preferably nitinol or another alloy).

Notably, each of the multifilament wires 24 follows a generally helical path between the first joint 36 and the second joint 38. As used herein, “helical” or “generally helical” means a path that resembles a helical or spiral path, and encompasses paths which may vary from a perfectly helical or spiral path. For example, the wires 24 may initially extend linearly as they emerge from the first joint 36, and again as they approach the second distal joint 38. Accordingly, the wire basket 22 is given a helical configuration through the spiral or helical formation of the individual multifilament wires 24.

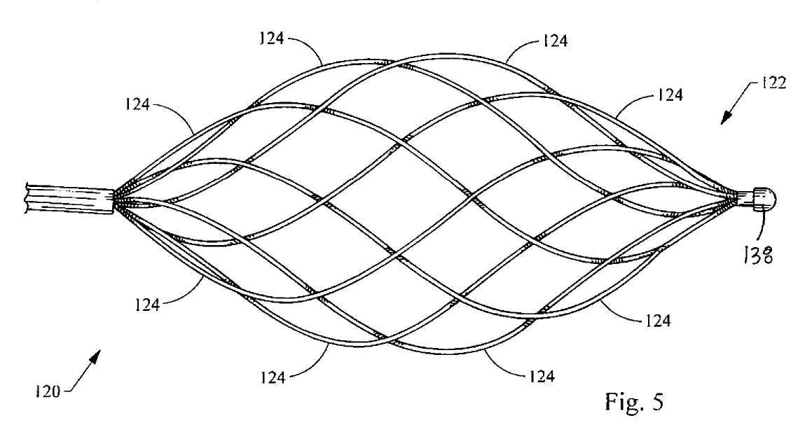

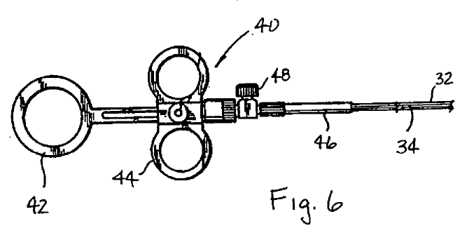

FIG. 4 depicts a perspective view of the medical device 20 and its helical wire basket 22, where the spiral path of the plurality of wires 24 can clearly be seen. It can also be seen that the wire basket 22 is constructed of four multifilament wires 24. It will be recognized by those skilled in the art that any number of wires may be used to form the basket 22. For example, FIG. 5 depicts a perspective view of another embodiment of the medical device 120 having a wire basket 122 formed out of eight multifilament wires 124. As with the prior embodiment, the medical device 120 includes a delivery cannula 132 for introducing the basket 122 into the body. The wire basket 122 is given a helical configuration through the spiral shaping of the multifilament wire 124, which are joined at their distal end to define an atraumatic distal tip 138.

It can also be seen in FIGS. 4 and 5 that each of the multifilament wires 24, 124 follow a helical path which turns counter-clockwise (when looking down the device 20 from the proximal end to the distal end). As seen in FIG. 2, the outer wires 28a-28f also turn in a counter-clockwise direction around the inner wire 26 (the proximal end being depicted to the left of the page in FIG. 2). The plurality of multifilament wires 24, 124 forming the wire basket 22, 122 exhibit the flexibility, stiffness and strength to traverse the anatomy and open a duct, while being able to perform lithotripsy on foreign bodies such as stones. In particular, this construction of the wire basket 22 improves the ability to dilate a duct over an elongated area and improve the capture and engagement of a stone from the side of the basket 22. At the same time, engagement of the stone is provided over an area which extends both longitudinally and circumferentially to improve crushing ability.

A method of performing lithotripsy is also provided in accordance with the teachings of the present invention. Generally, the delivery cannula 32 having the wire basket 22 retracted therein is advanced beyond the target foreign body. The basket 22 is advanced out of the delivery cannula 32 by pushing on the control member 34 or by retracting the delivery cannula 32. The device 20 is retracted proximally and the basket 22 is manipulated to capture the foreign body. The control member 34 is then retracted to entrap the foreign body and tighten the multifilament wires 24 around the foreign body. Then, the delivery cannula 32 and control member 33 are cut using wire cutters and a reinforced lithotripsy cable, such as the Wilson-Cook® Conquest TTC™, is advanced over the delivery cannula 32 until the cable reaches the basket in the duct. The control member 34 and sheath 32 are then connected to a lithotripter handle, such as the Wilson-Cook® Soehendra® Lithotriptor Handle, which utilizes a hand crank to wind up the control member 34 and retract the member relative to the lithotripsy cable for collapsing the wire basket 22 and fracturing the stone.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A medical device for performing lithotripsy on a foreign body within a patient, the medical device comprising:
a wire basket constructed from a plurality of multifilament wires, the wire basket configured to engage and crush the foreign body; and
the plurality of multifilament wires each following a generally helical path to give the wire basket a generally helical configuration.

2. The medical device of claim 1, wherein the plurality of multifilament wires are joined together at a first location and joined together at a second location, the second location being axially spaced from the first location, the wire basket defined between the first and second locations.

3. The medical device of claim 2, wherein a distal tip of the wire basket is formed at the second location.

4. The medical device of claim 3, wherein the plurality of multifilament wires are joined at the second location by solder, the solder forming an atraumatic distal tip.

5. The medical device of claim 1, wherein the wire basket is operable between a collapsed configuration for introduction into the patient and an expanded configuration for capturing the foreign body.

6. The medical device of claim 1, wherein each multifilament wire includes at least three wires stranded together.

7. The medical device of claim 1, wherein each multifilament wire includes an inner wire surrounded by a plurality of outer wires.

8. The medical device of claim 7, wherein the plurality of outer wires are stranded together about the inner wire.

9. The medical device of claim 7, wherein the inner wire is constructed of a shape memory material.

10. The medical device of claim 9, wherein the wire basket is operable between a collapsed configuration for introduction into the patient and an expanded configuration for capturing the foreign body, and wherein the inner wire is formed to follow the generally helical path in the expanded configuration.

11. The medical device of claim 7, wherein the outer wires are constructed of stainless steel.

12. The medical device of claim 1, further comprising a control member connected to the wire basket and extending proximally therefrom.

13. The medical device of claim 12, wherein the control member is formed by the plurality of multifilament wires.

14. The medical device of claim 12, further comprising a delivery cannula defining a delivery lumen, and wherein the control member is slidably disposed within the delivery lumen.

15. The medical device of claim 14, wherein the wire basket is operable between a collapsed configuration for introduction into the patient and an expanded configuration for capturing the foreign body, and wherein the wire basket is received within the delivery lumen in the collapsed configuration.

16. The medical device of claim 14, further comprising a handle having a first member slidable relative to a second member, the first member connected to the control member, the second member connected to the delivery cannula, the handle controlling the relative movement of the control member and delivery cannula.

17. A medical device for retrieving a foreign body from within a patient, the medical device comprising:
a wire basket constructed of a plurality of multifilament wires joined at a first location and joined at a second location axially spaced from the first location, the wire basket configured to engage and crush the foreign body, the plurality of multifilament wires the plurality of wires following a helical path between the first and second locations;
a control member connected to the wire basket at the second location; and
a delivery cannula defining a delivery lumen, wherein the control member is slidably disposed within the delivery lumen.

18. The medical device of claim 17, wherein the control member is formed by the plurality of multifilament wires.

19. The medical device of claim 17, wherein the wire basket is operable between a collapsed configuration for introduction into the patient and an expanded configuration for capturing the foreign body, and wherein the wire basket is received within the delivery lumen in the collapsed configuration.

20. The medical device of claim 19, wherein the delivery cannula is configured such that retraction of the wire basket into the delivery lumen causes the wire basket to crush the foreign body as the wire basket transitions from the expanded configuration to the collapsed configuration.

21. The medical device of claim 17, further comprising a handle having a first member slidable relative to a second member, the first member connected to the control member, the second member connected to the delivery cannula, the handle controlling the relative movement of the control member and delivery cannula.

22. A method of performing lithotripsy on a foreign body within a bodily lumen of a patient, the method comprising the steps of:
providing a wire basket connected to a control member, the wire basket constructed from a plurality of multifilament wires each following a generally helical path to give the wire basket a generally helical configuration, the control member extending proximally from the wire basket;
providing a delivery cannula defining a delivery lumen, the control member being slidably disposed within the delivery lumen such that the wire basket is operable between a collapsed configuration within the delivery lumen and an expanded configuration outside of the delivery lumen;
positioning the wire basket and delivery cannula proximate the foreign body in the collapsed configuration;
translating the control member relative to the delivery cannula such that the wire basket assumes the expanded configuration;
manipulating the control member to capture the foreign body within the wire basket from a side of the wire basket; and
causing the wire basket to assume its collapsed configuration to crush the foreign body.

23. The method of claim 22, wherein the causing step includes translating the control member relative to the wire basket.

24. The method of claim 22, wherein the wire basket dilates the bodily lumen in the expanded configuration.

25. The method of claim 22, further comprising the steps of providing a reinforced lithotripsy cable and translating the cable over the delivery cannula.

26. The method of claim 22, wherein the causing step includes translating the control member relative to the reinforced lithotripsy cable.
